(19) United States
(12) Patent Application Publication
(10) Pub. No.: US 2026/0148840 A1
(43) Pub. Date: May 28, 2026

(54) HYBRID SERVER-SIDE CLIENT-SIDE RENDERING OF MEDICAL IMAGES

(71) Applicant: FOVIA, INC., Palo Alto, CA (US)

(72) Inventors: Abhijit DESHMUKH, Palo Alto, CA (US); Noah BRICKMAN, Palo Alto, CA (US); David C. WILKINS, Palo Alto, CA (US); Georgiy BUYANOVSKIY, Palo Alto, CA (US); Kevin KREEGER, Palo Alto, CA (US)

(21) Appl. No.: 19/395,911

(22) Filed: Nov. 20, 2025

Related U.S. Application Data
(60) Provisional application No. 63/724,136, filed on Nov. 22, 2024.

(51) Int. Cl. G16H 30/40; G06F 3/14; G16H 30/20; G16H 40/67

(52) U.S. Cl. CPC G16H 30/40; G06F 3/14; G16H 30/20; G16H 40/67

(57) ABSTRACT

Methods, systems, and computer-readable medium for rendering medical images are provided. In one example, a method includes creating a first rendered image from a set of scan data in response to a request from a client device. The first rendered image is then sent to the client device for display based on a subset of the set of scan data, and after sending the first rendered image to the client device for display, sending the set of scan data to the client device for use to render additional images based on the set of scan data (e.g., rendering additional images from the scan data locally at the client). The set of scan data can be streamed or sent to the client device in the background, during low network activity times, etc., in parallel or after sending the requested medical image.

### CROSS-REFERENCE TO RELATED APPLICATION

This application claims benefit to U.S. Provisional Application Ser. No. 63/724,136, filed Nov. 22, 2024, entitled “HYBRID SERVER-SIDE CLIENT-SIDE RENDERING OF MEDICAL IMAGES,” the contents of which is hereby incorporated by reference in its entirety for all purposes.

### FIELD

This disclosure relates generally to computer graphics and the rendering of medical images, and in one example to a hybrid server-side client-side rendering of medical images.

### BACKGROUND

Display workstations for radiologists have demanding performance characteristics. Radiologists typically visualize scans (such as Xray, Computed Tomography, Magnetic Resonance Imaging, Ultrasound and more) that have many large images (e.g., large dataset sizes). Recently, radiologists have been performing more of their work remotely, either centralized reading locations for hospital/imaging center groups with multiple distributed sites, or complete tele-radiology of scans from anywhere in the world. For this reason, there is a recent trend in radiology display systems to be run from the cloud or at least from remote servers where the scanned data is stored.

### SUMMARY

Methods, systems, and computer-readable medium for rendering medical images are provided herein. In one example, a computer-implemented method for rendering medical images includes creating a first rendered image from a set of scan data (e.g., on a server device) in response to a request from a client device. The first rendered image is then sent to the client device for display based on a subset of the set of scan data, and after sending the first rendered image to the client device for display, sending the set of scan data to the client device for use to render additional images based on the set of scan data (e.g., rendering additional images from the scan data locally at the client). For instance, the set of scan data can be streamed or sent to the client device in the background, during low network activity times, etc., in parallel or after sending the requested rendered image. If a second request is received for a medical image prior to sending the set of scan data, the method can further render the second medical image and send to the client. From the client perspective, when a user requests additional images, adjusts any rendering parameters (e.g., from mouse or keyboard actions), or the like, if sufficient scan data required to create the new display image is present on the client computer then client-side rendering is utilized to render a display image and put it on screen. If sufficient scan data is not yet present the system utilizes server-side rendering and sends the render parameters to the server (where the entire dataset is already loaded) and a server-side image is rendered and sent to the client for display.

The described hybrid rendering methods, systems, and computer-readable medium for rendering medical images provides a user with a first requested image more quickly than with client-side rendering (as there is no delay for the entire scan dataset to be received, e.g., due to high network traffic, size, and the like) and further offloads computing resources from the server to the client device once the scan data is communicated to the client for further image renderings.

### FIGURES

FIG. 1 illustrates an exemplary system diagram of a hybrid server-side client-side rendering system for rendering medical images.

FIG. 2 illustrates an exemplary timing diagram between various modules of an exemplary system for hybrid server-side client-side rendering system.

FIG. 3 illustrates an exemplary timing diagram between various modules of an exemplary system for hybrid server-side client-side rendering system.

FIG. 4 illustrates an exemplary process for hybrid server-side client-side rendering.

### DETAILED DESCRIPTION

As described herein, exemplary processes, systems, and computer-readable storage medium for client-side server-side hybrid rendering of medical images are provided. Various embodiments described herein may be carried out by computer devices, medical imaging systems, and computer-readable non-transitory storage medium comprising instructions for carrying out the described methods.

Display workstations for radiologists have demanding performance characteristics. Radiologists typically visualize scans (such as Xray, Computed Tomography, Magnetic Resonance Imaging, Ultrasound and more) that have many large images (e.g., large dataset sizes). Recently, radiologists have been performing more of their work remotely, either centralized reading locations for hospital/imaging center groups with multiple distributed sites, or complete tele-radiology of scans from anywhere in the world. For this reason, there is a recent trend in radiology display systems to be run from the cloud or at least from remote servers where the scanned data is stored.

Radiology images are not typically displayed directly as they are scanned. The radiologists often control the brightness and contrast of the image to highlight different abnormalities, and they also zoom the image and pan to an interesting portion of the image. If the study contains multiple images the exact image to display can be selected and 3D rendering s of scans such as CT and MRI are common. For this reason, the actual displayed images to the radiologist are rendered for display on the radiologist viewing workstation.

When the display of the images for the radiologist to read and location of the original scanned data are at 2 different places, there are 2 basic configurations for getting the data, rendering it, and finally displaying it to the physician.

The first method is typically called server-side rendering. In this configuration, the data is loaded in a server computer close to where the original scanned data is stored. Images are rendered on that server and then the resulting displayable images are sent (streamed) over a network to the client software on a machine in front of the radiologist for final display. Usually the client software will include features to capture mouse or keyboard controls from the user and adjust the parameters by which the image is to be rendered, send these changes to the server and the server creates a new image to then send (stream) over the client for final display.

The second configuration is typically called client-side rendering. In this configuration, the data must be sent from the server where the data is stored over the network to the client computer for use by the radiologist. After the data is sent over then the rendering process occurs on the client computer, and the final image is displayed on the client computer. When the radiologist requests viewing adjustments (again using mouse or keyboard input) the rendering parameter changes stay on the client. A new image of the original scanned data is created right there and displayed to the physician.

There are tradeoffs between the two methods. For instance, server-side rendering can display the first image to a user much more quickly than client-side rendering because the entire dataset does not need to be transferred across the network before the first image is generated (and, especially for studies with multiple images each displayable image is much smaller than the entire dataset, but even for Xray single image it can be smaller because the displayed image is still smaller than the original scan).

On the other hand, client-side rendering is less susceptible to network slowdown and glitches because once the data is all transferred there is no need to communicate back and forth between the client and server, which may cause delays. The total amount of data transfer is sometimes better for server-side rendering and sometimes better for client-side rendering depending on the total size of the original scanned data, the number of display images the physician wants to review to complete the case, and the like.

Another tradeoff between the two configurations that has recently become more important is server-costs and scalability. When the data storage location and servers grow to support a very large number of physician users, the costs to allocate and configure and bring online a large number of server-side rendering computers grows. With client-side rendering, each new user brings with them a compute resource to perform their rendering operations, thereby reducing sever-side resources and costs.

In one example provided herein, a process is provided that uses features of each of these two configurations in a hybrid approach that brings along certain advantages of each configuration. In one example, the following process includes:
1. The data from an original scan is loaded into the server close to where the original scanned data is stored.
2. This server creates a first rendered image of the data.
3. The rendered image of the data is sent to the client for display.
4. In the background, e.g., when the network is not busy, the original scanned data is streamed over to the client.
5. On the client machine, the first rendered image from the server is displayed.
6. When a physician/user adjusts any rendering parameters (e.g., from mouse or keyboard actions), if all the data required to create the new display image is present on the client computer then client-side rendering is utilized to render a display image and put it on the screen. If all the data is not yet present the system utilizes server-side rendering and send the render parameters over to the remote server (where the entire dataset is already loaded) and a server-side image is rendered and sent across the network to the client for display.

This is different than a hybrid rendering approach where the system utilizes server-side rendering exclusively until all the data has arrived at the client computer. In this example described above, for each requested image the client checks if the required data is present. The required data may be a subset of the entire dataset and thus it may utilize client-side rendering more quickly than requesting from the server. In fact, some images may take advantage of client-side rendering and subsequent requested images may have to revert to server-side because they require more data than has transferred so far.

Additionally, in some examples, a common rendering parameters data structure that is shared server-side and client side since the rendering may toggle back and forth. This allows for consistency in image appearance between the two.

Finally, in another example, processes and methods for optimizing the transfer of the data are provided. For example, the exemplary process and system may utilize compressed data to transfer the original scanned images. In some examples, this may be achieved using multiple network connections (e.g., websockets) to better utilize any available network bandwidth.

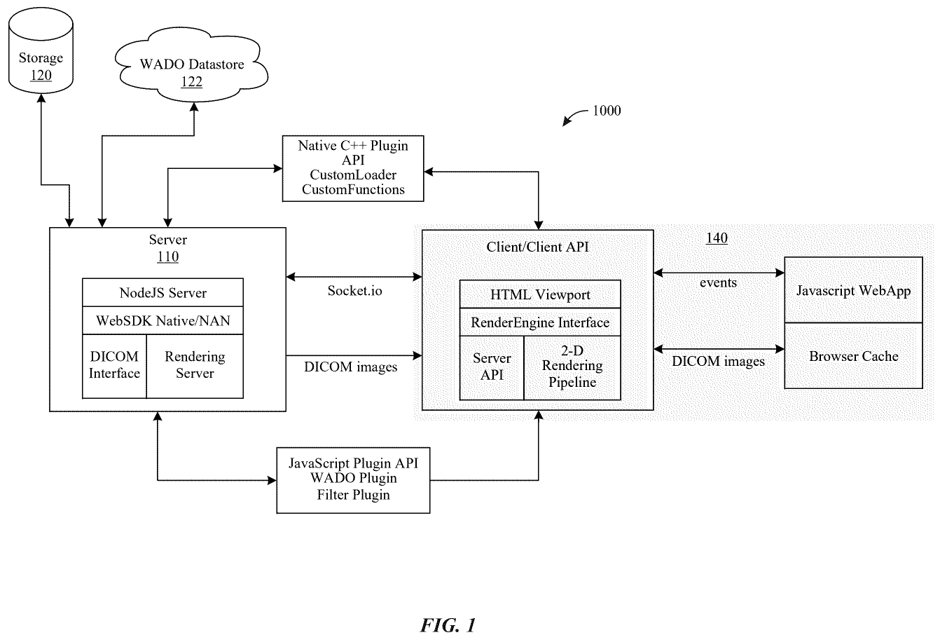

FIG. 1 illustrates an exemplary system diagram of a hybrid server-side client-side rendering system 1000 for rendering medical images. In this example, system 1000 includes a server 110, which may communicate with one or more of a storage 120 and datastore 122, e.g., which may store medical scan data, WADO Datastore, etc. Server 110 may include various components such as a DICOM interface, rendering server, and the like, to both render images based on scan data and also transmit scan data to client device 140. The client and server in this example, may communicate in various ways, including Native C++ Plugin APIs, JavaScript Plugin APIs, sockets, etc.

Client device 140 includes, in one example, client APIs 142, which includes modules for viewing images and rendering images, e.g., an HGML viewport, render engine, rendering pipelines, etc. These modules may interact with the server as described herein to receive serve side rendered images as well as scan data. Further, the modules may interact with the server side to request additional rendered images, e.g., based on adjusted parameters or additional views requested by the user prior to receiving the scan data. In some examples, client-side web applications and browsers may be used to view the rendered images, whether rendered client-side or server-side.

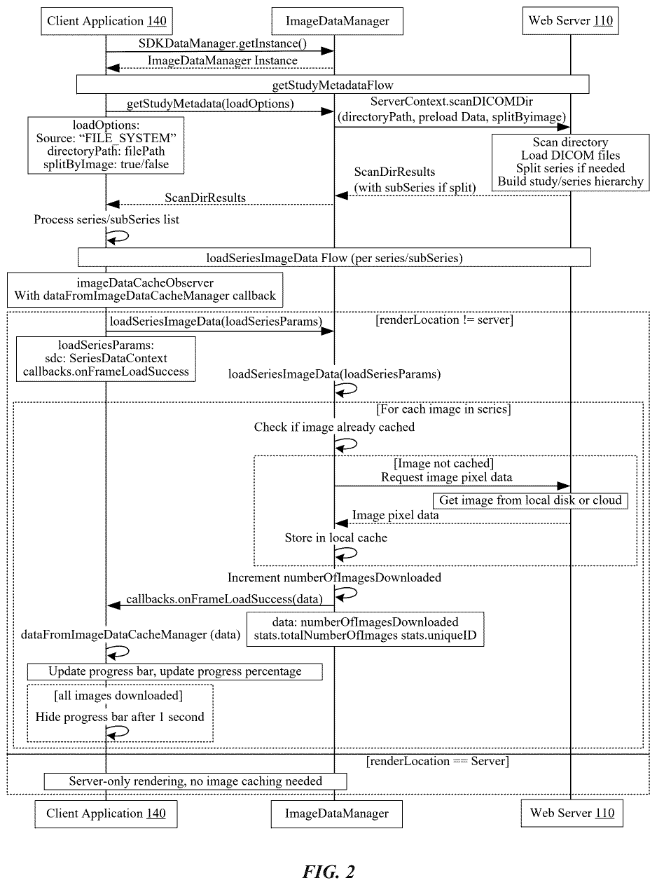

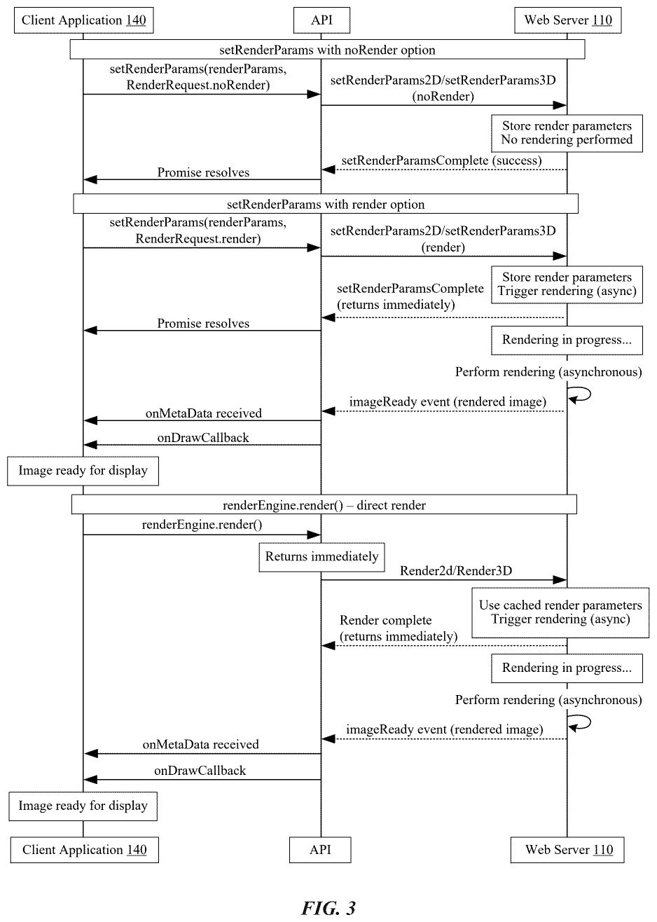

FIGS. 2 and 3 illustrate exemplary timing diagrams between various modules of exemplary system 1000 for hybrid server-side client-side rendering, including loading scan data and a rendering pipeline, respectively. Initially, a user at a client device 140 requests to view an image. This request, along with various parameters associated with the request as to the particular image the user has requested, are communicated to server 110. In response to the request, server 110 loads in associated scan data associated with the requested image and renders the first image that was requested. The rendered image is then communicated to client device 140. Additionally, server 110 initiates and begins to send the scan data from which the image was rendered to client device 140 (for potential future rendering(s) of images based on the scan data as requested by client device 140).

If a user requests a new image, e.g., changing views, slices, parameters, etc. of the original image, and the full set of scan data has been transferred to client device 140, client device can render the image locally (saving the latency of requesting a new image rendered at server 110 and communicated to client device 140). If the scan data has not been fully or sufficiently transferred to client device 140 when a request for a new image is received, a new request for the new image can be sent to server 120 for rendering from the scan data as described above. It should be noted that although references are made herein to rendering single images, e.g., single image DICOM, it also contemplated for use with volume rending where multiple images would be generated by the server and sent to the client for viewing prior to fully sending the scan data to the client for future rending processes.

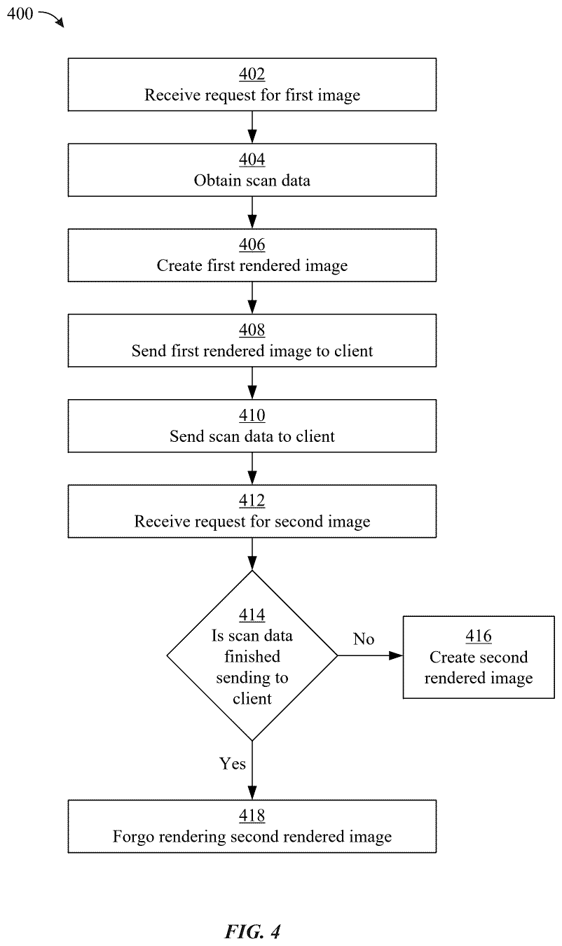

FIG. 4 illustrates an exemplary process 400 for hybrid server-side client-side rendering. A client device (e.g., device 140) requests a first image for view at 402. In response, a server (e.g., server 110) obtains associated scan data (e.g., from storage 120, WADO datastore 122, etc.). A first rendered image is then created at 406 and communicated to the client device at 408 for viewing, e.g., via an API, browser viewer, etc.). Additionally, the server initiates sending the associated scan data to the client at 410. The scan data can be sent at least partially in parallel with the first rendered image or thereafter, e.g., in the background or when network traffic is low (e.g., below a threshold). When a subsequent request for an image is made the client may have the required data locally (even if all of the scan data is not yet received) and the client can render the image. If the user requests a subsequent new image and the required scan data has not been received at the client a request to the server to render may be made. In other words, while the scan data is being communicated or streamed to the client, the system may switch back and forth between rendering images on the server or client (depending on whether sufficient data has been received by the client). Once the scan data has been completely sent and received by the client, the client can thereafter render additional images locally.

In response to a request for second image at 412, if it is determined at 414 that the scan data has not been completely transferred to the client (or at least not sufficient scan data to render the requested image), the server can create a second rendered image at 416 and thereafter communicate to the client. If, however, it is determined that the scan data has been fully or sufficiently sent to and received by the client, the server will forgo rendering a second image at 418 (and/or the client will forgo requesting the image from the server). In some examples, the client device may determine locally if the scan data has been sufficiently received (even if not fully received, i.e., enough has been received to render the requested image) to render the image, and if so, not request additional images from the server (and render locally).

Various exemplary embodiments are described herein. Reference is made to these examples in a non-limiting sense. They are provided to illustrate more broadly applicable aspects of the disclosed technology. Various changes may be made, and equivalents may be substituted without departing from the true spirit and scope of the various embodiments. In addition, many modifications may be made to adapt a particular situation, material, composition of matter, process, process act(s) or step(s) to the objective(s), spirit or scope of the various embodiments. Further, as will be appreciated by those with skill in the art, each of the individual variations described and illustrated herein has discrete components and features that may be readily separated from or combined with the features of any of the other several embodiments without departing from the scope or spirit of the various embodiments. All such modifications are intended to be within the scope of claims associated with this disclosure.

1. A computer-implemented method for rendering medical images, the method comprising:
at an electronic device including one or more processors and memory:
creating a first rendered image from a set of scan data in response to a request from a client device;
sending the first rendered image to the client device for display based on a subset of the set of scan data; and
after sending the first rendered image to the client device for display, sending the set of scan data to the client device for use to render additional images based on the set of scan data.

2. The computer-implemented method of claim 1, further comprising:
receiving a request for a second rendered image before the set of scan data is completely sent to the client device, the render request comprising render parameters;
creating a second rendered image from the set of scan data; and
sending the second rendered image to the client device.

3. The computer-implemented method of claim 1, further comprising sending the set of scan data to the client device when network traffic is below a threshold.

4. The computer-implemented method of claim 1, further comprising initiating sending the set of scan data to the client device in response to receiving the request from the client device and completing the sending of the set of scan data after sending the first rendered image.

5. A computer-implemented method for rendering medical images, the method comprising:
at an electronic device including one or more processors and memory:
requesting a first rendered image from a set of scan data, wherein at least some of the set of scan data has been received;
determining if sufficient scan data of the set of scan data is present locally to render the image;
if sufficient scan data is present locally, rendering the image based on the scan data received; and
if sufficient scan data is not present locally, requesting the first image from the server, and thereafter receiving the first image from the server.

6. A system, comprising:
one or more processors;
a memory; and
one or more programs, wherein the one or more programs are stored in the memory and configured to be executed by the one or more processors, the one or more programs including instructions for:
creating a first rendered image from a set of scan data in response to a request from a client device;
sending the first rendered image to the client device for display based on a subset of the set of scan data; and
after sending the first rendered image to the client device for display, sending the set of scan data to the client device for use to render additional images based on the set of scan data.

7. The system of claim 6, further comprising instructions for:
receiving a request for a second rendered image before the set of scan data is completely sent to the client device, the render request comprising render parameters;
creating a second rendered image from the set of scan data; and
sending the second rendered image to the client device.

8. The system of claim 6, further comprising instructions for sending the set of scan data to the client device when network traffic is below a threshold.

9. The system of claim 6, further comprising instructions for initiating sending the set of scan data to the client device in response to receiving the request from the client device and completing the sending of the set of scan data after sending the first rendered image.

10. A non-transitory computer-readable storage medium storing one or more programs configured to be executed by one or more processors of an electronic device, the one or more programs including instructions for:
creating a first rendered image from a set of scan data in response to a request from a client device;
sending the first rendered image to the client device for display based on a subset of the set of scan data; and
after sending the first rendered image to the client device for display, sending the set of scan data to the client device for use to render additional images based on the set of scan data.

11. The non-transitory computer-readable storage medium of claim 10, further comprising instructions for:
receiving a request for a second rendered image before the set of scan data is completely sent to the client device, the render request comprising render parameters;
creating a second rendered image from the set of scan data; and
sending the second rendered image to the client device.

12. The non-transitory computer-readable storage medium of claim 10, further comprising instructions for sending the set of scan data to the client device when network traffic is below a threshold.

13. The non-transitory computer-readable storage medium of claim 10, further comprising instructions for initiating sending the set of scan data to the client device in response to receiving the request from the client device and completing the sending of the set of scan data after sending the first rendered image.
